(19) United States
(12) Patent Application Publication
(10) Pub. No.: US 2017/0101365 A1
(43) Pub. Date: Apr. 13, 2017

(54) AZIDOALKYLAMINE SALTS AND THEIR USE AS INTERMEDIATES

(71) Applicant: DIPHARMA FRANCIS S.R.L., Baranzate (MI) (IT)

(72) Inventors: Pietro ALLEGRINI, Baranzate (MI) (IT); Gabriele RAZZETTI, Baranzate (MI) (IT); Dario PASTORELLO, Baranzate (MI) (IT)

(73) Assignee: DIPHARMA FRANCIS S.R.L., Baranzate (MI) (IT)

(21) Appl. No.: 15/314,082

(22) PCT Filed: May 26, 2015
(86) PCT No.: PCT/IB2015/053932

§ 371 Date: Nov. 27, 2016

(30) Foreign Application Priority Data
May 27, 2014 (IT) MI2014A000964

(51) Int. Cl. C07C 247/04; C07D 498/04; C07C 59/265; C07C 209/82

(52) U.S. Cl. CPC C07C 247/04; C07C 209/82; C07D 498/04; C07C 59/265

(57) ABSTRACT

The present invention relates to azidoalkylamine salts of formula (I) with organic acids, a process for their preparation, and their use as intermediates in the preparation of active pharmaceutical ingredients or polymers, or as spacers useful in organis synthesis. NH₂—(CH₂)n-N₃ (I)

### FIELD OF INVENTION

The present invention relates to azidoalkylamine salts with organic acids, a process for their preparation, and their use in the preparation of active pharmaceutical ingredients, polymers or spacers useful in organic synthesis.

### PRIOR ART

Azidoalkylamines, such as 4-azidobutylamine, are compounds which have long been used in organic synthesis and are universally acknowledged to be useful in the preparation of active pharmaceutical ingredients, in polymer synthesis, or more generally as spacers in the preparation of organic compounds. In particular, 4-azidobutylamine is a compound used to prepare active ingredients with an antibiotic action, such as those belonging to the macrolide class, in particular solithromycin.

As is well known to the skilled person, azidoalkylamines, and in particular 4-azidobutylamine, due to the presence of an azide group, are compounds with explosive characteristics which are difficult to handle and unstable in physicochemical terms; said compounds are also difficult to obtain in pure form.

The difficulty of stabilising azidoalkylamines, especially 4-azidobutylamine, and the problems involved in handling those compounds, create significant problems with the supply of the desired product to third parties. In order to eliminate said problems, manufacturers of azidoalkylamines, especially 4-azidobutylamine, have therefore had to take special precautions during their manufacture and transport to date; hauliers must hold special permits from the authorities, and the vehicles used must be specifically dedicated and comply with specific transport conditions.

There is consequently a need to find a solution to the problems set out above. Against this background, the present invention provides azidoalkylamine salts that eliminate the drawbacks and problems of azidoalkylamines, in particular the stability and explosion problems reported above.

### SUMMARY OF THE INVENTION

Disclosed are salts of a compound of formula (I)

NH₂—(CH₂)n-N₃ (I)

with an organic acid wherein n is an integer from 1 to 15, preferably in crystalline, amorphous or solvated form, a process for their preparation, and their use as intermediates in the preparation of active pharmaceutical ingredients, in particular solithromycin.

BRIEF DESCRIPTION OF FIGURES AND ANALYSIS METHODS

4-azidobutylamine salts have been characterised by differential scanning calorimetry (DSC), and the DSC pattern of 4-azidobutylamine, as such, is set out below.

The DSC patterns were acquired with a Mettler-Toledo DSC 822e differential scanning calorimeter under the following operating conditions:

gold crucible, temperature range 30-400° C. with heating rate of 4-10° C/min, closed in inert nitrogen atmosphere.

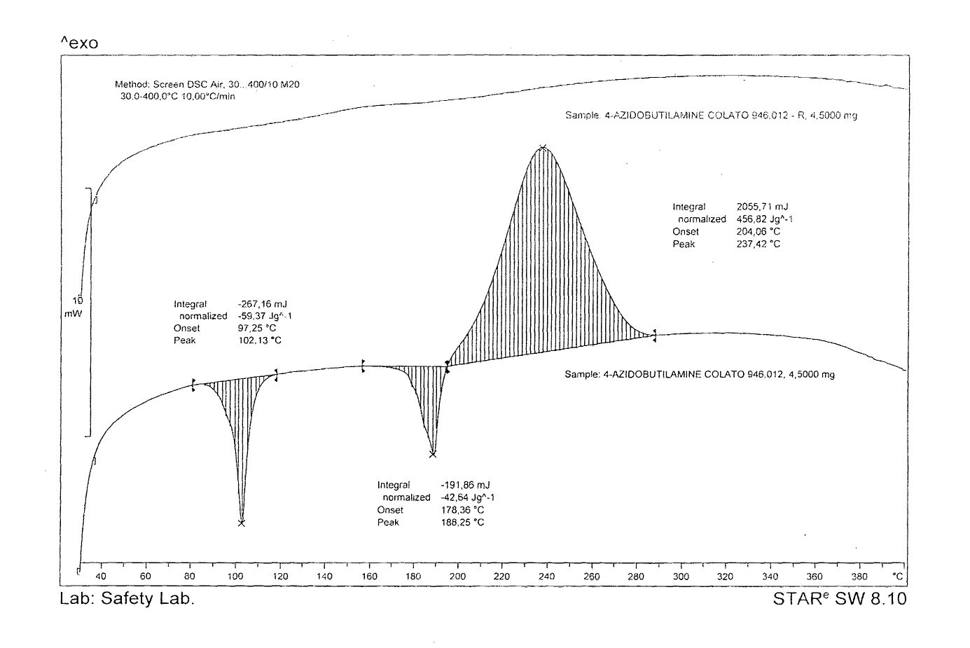

FIG. 1: DSC pattern of 4-azidobutylamine cholate

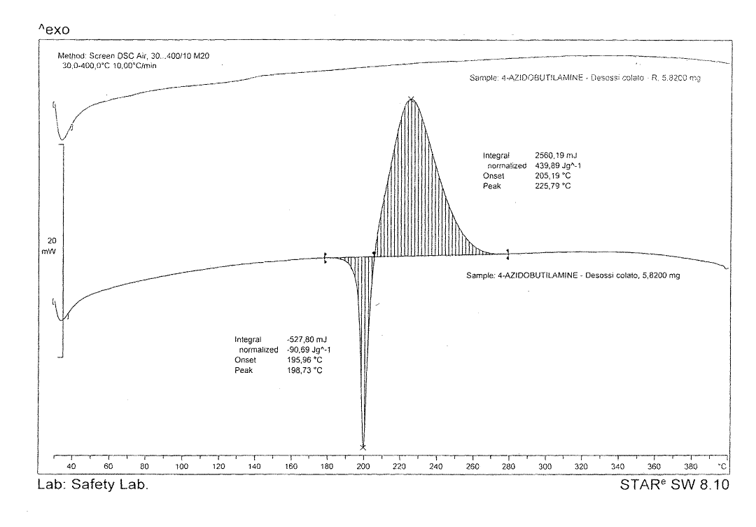

FIG. 2: DSC pattern of 4-azidobutylamine deoxycholate

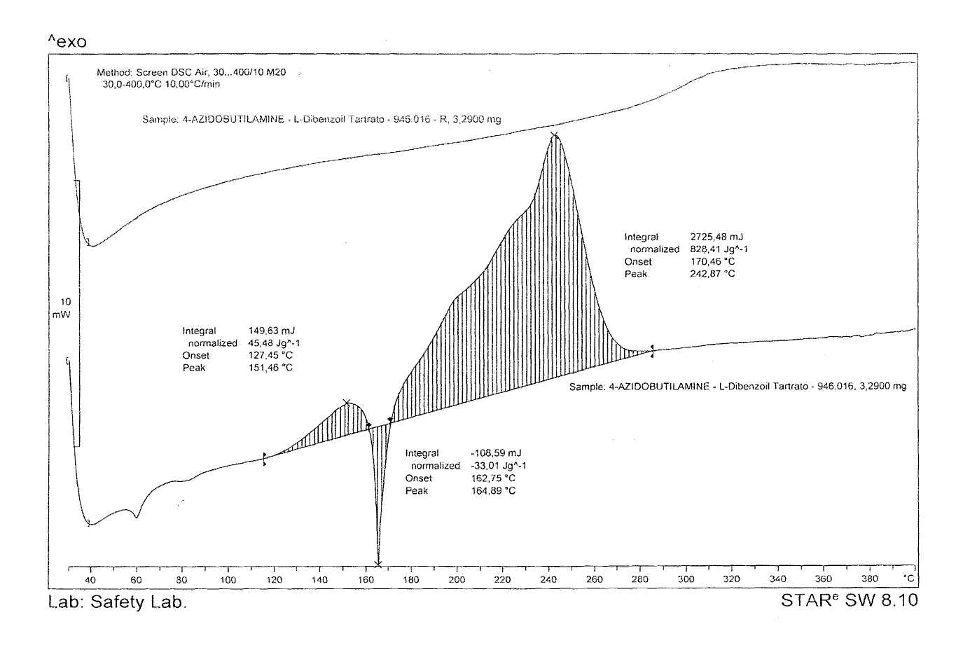

FIG. 3: DSC pattern of 4-azidobutylamine L-dibenzoyl tartrate

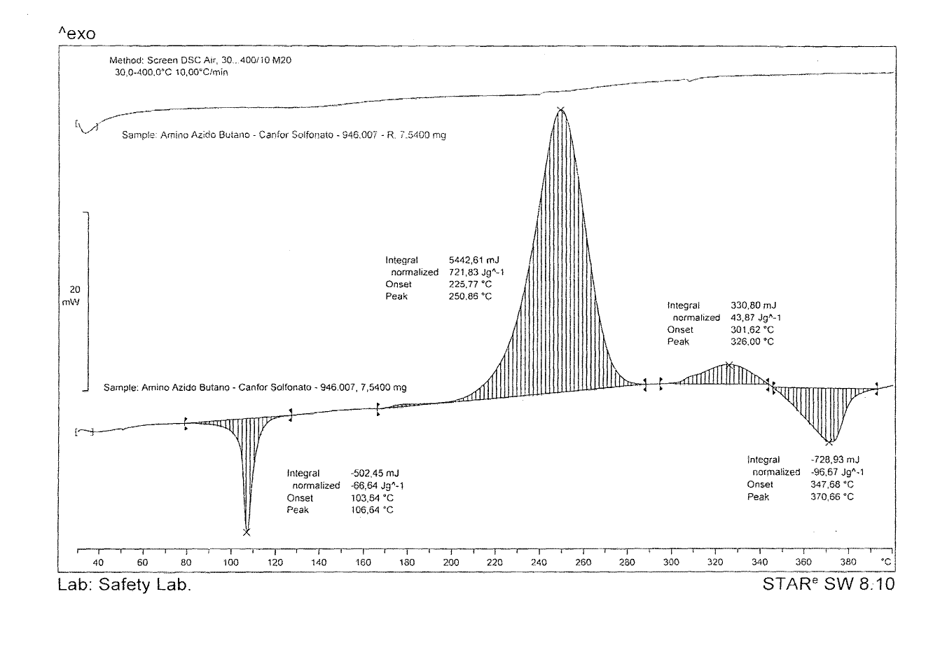

FIG. 4: DSC pattern of 4-azidobutylamine camphorsulphonate

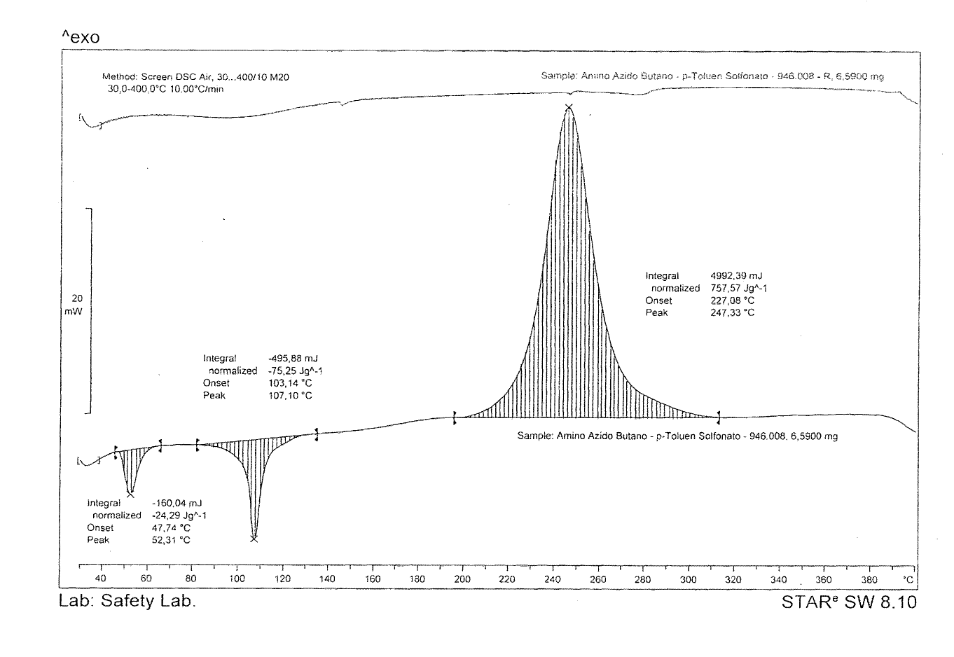

FIG. 5: DSC pattern of 4-azidobutylamine p-toluenesulphonate

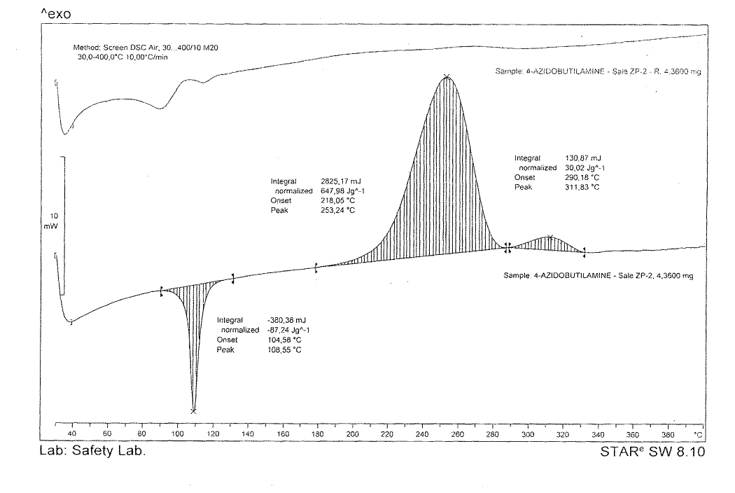

FIG. 6: DSC pattern of 4-azidobutylamine 4-phenylbutyl-2-carboxyethyl-phosphinate

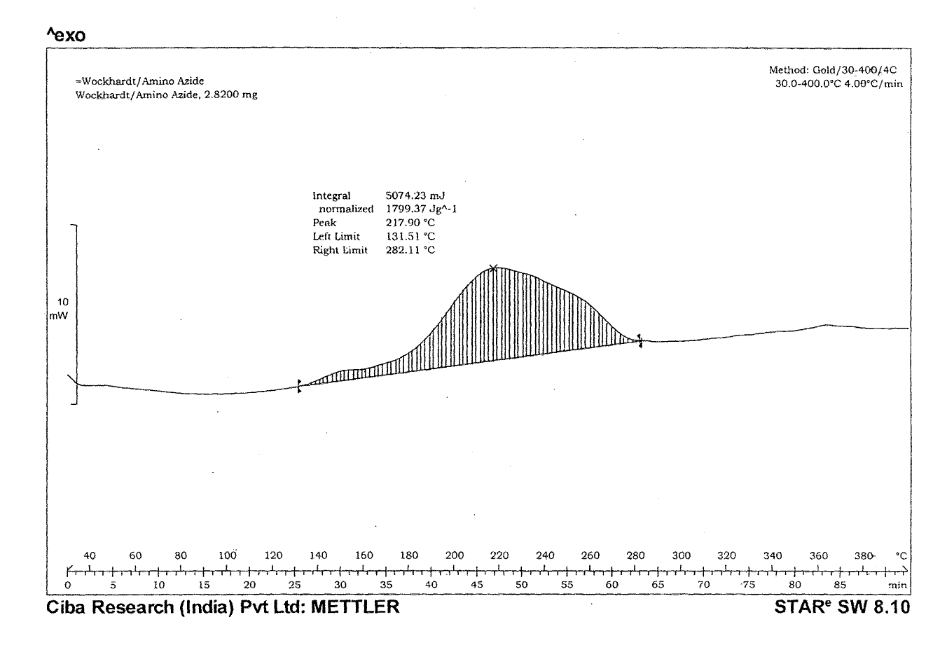

FIG. 7: DSC pattern of 4-azidobutylamine.

### DETAILED DESCRIPTION OF THE INVENTION

The subject of the present invention is a salt of a compound of formula (I)

NH₂—(CH₂)n-N₃ (I)

with an organic acid, wherein n is an integer from 1 to 15, preferably in crystalline, amorphous or solvated form.

In a compound of formula (I) n is preferably an integer from 2 to 6, more preferably from 3 to 5, in particular 4.

An organic acid can be a carboxylic acid, a sulphonic acid, a phosphinic acid or a phosphonic acid.

A carboxylic acid, which can be aliphatic or aromatic, saturated or unsaturated, acyclic or cyclic, is selected, for example, from the group comprising an optionally substituted monocarboxylic, dicarboxylic or tricarboxylic acid.

A monocarboxylic acid is typically selected from the group comprising a cholanic acid, such as cholic acid, deoxycholic acid, chenodeoxycholic acid, hyodeoxycholic acid and ursodeoxycholic acid; pantoic acid; pantothenic acid; folic acid; a fatty acid, such as palmitic acid, stearic acid, oleic acid, linoleic acid, linolenic acid, butyric acid, valerianic acid, caproic acid, enanthic acid, caprylic acid, pelargonic acid, caprinic acid, lauric acid, myristic acid, margaric acid, behenic acid, lignoceric acid, cerotic acid, montanic acid, melissic acid, lacceroic acid, palmitoleic acid, elaidinic acid, vaccenic acid, gadoleic acid, cetoleic acid, erucic acid, nervonic acid, rumenic acid, stearidonic acid, arachidonic acid, timnodonic acid, clupanodonic acid or cervonic acid; glycolic acid; hyaluronic acid; acetylsalicylic acid; salicylic acid. A monocarboxylic acid is preferably a cholanic acid, in particular cholic acid or deoxycholic acid.

A dicarboxylic acid is typically selected from the group comprising tartaric acid, dibenzoyltartaric acid, fumaric acid, succinic acid, adipic acid, malic acid, maleic acid and oxalic acid. A dicarboxylic acid is preferably dibenzoyltartaric acid.

A tricarboxylic acid is typically citric acid.

A sulphonic acid, which can be aliphatic or aromatic, is typically methanesulphonic, camphorsulphonic or para-toluenesulphonic acid. A sulphonic acid is preferably camphorsulphonic or para-toluenesulphonic acid.

A phosphinic acid can be any commercially known phosphinic acid, preferably 4-phenylbutyl-2-carboxyethyl-phosphinic acid.

Preferred examples of novel salts of a compound of formula (I), according to the invention, are 4-azidobutylamine cholate, 4-azidobutylamine p-toluenesulphonate, 4-azidobutylamine camphorsulphonate, 4-azidobutylamine deoxycholate, 4-azidobutylamine L-dibenzoyl tartrate and 4-azidobutylamine 4-phenylbutyl-2-carboxyethyl-phosphinate.

A further subject of the present invention is a process for the preparation of a salt of the compound of formula (I), as defined above, comprising reacting a compound of formula (I), as defined above, with an organic acid, in the presence of a solvent if appropriate.

Said reaction is preferably conducted by a process comprising:
a) forming a solution of a compound of formula (I) in a solvent;
b) adding an organic acid to the resulting solution to obtain a precipitate;
c) recovering the salt of the compound of formula (I) thus obtained.

A compound of formula (I) used as starting material in the process described above is commercially available, and is preferably 4-azidobutylamine.

A solvent according to the process reported above is typically a solvent wherein a compound of formula (I) is miscible, for example selected from the group comprising a straight or branched, cyclic or acyclic ether, such as diethyl ether or methyl tert-butyl ether; a C₁-C₅ alkyl ester, typically ethyl or methyl acetate; a chlorinated solvent such as dichloromethane or an aromatic hydrocarbon such as toluene. The solvent is preferably methyl tert-butyl ether.

The concentration of a compound of formula (I) in the solution at step a) typically ranges between about 5 and about 20% w/w, preferably around 8-10% w/w.

The ratio between the organic acid and the compound of formula (I) typically ranges between about 1:1 and about 1.5:1, preferably around 1.03:1.

An organic acid is typically added to the solution at a temperature ranging between about 0 and about 30° C., preferably at room temperature.

If desired, to facilitate the formation of the precipitate in step b), an organic acid can be added to the solution by cooling the dispersion, for example to a temperature ranging between 0 and 10° C.

The salt of a compound of formula (I) can typically be recovered by methods known to the skilled person, such as centrifugation or filtration, for example through a Büchner filter.

The dimension of the crystals of a salt of a compound of formula (I) thus obtained typically ranges between about 50 and 250 μm, and if desired, said dimension can be further reduced by micronisation or fine grinding.

A salt of a compound of formula (I) with an organic acid obtained by the process according to the present invention, in particular a 4-azidobutylamine salt, has a purity equal to or greater than 99.8%, preferably exceeding 99.9%.

The salts of a compound of formula (I) with an organic acid, as defined above, are more stable in physicochemical terms than 4-azidobutylamine, as the skilled person can realise from the DSC patterns. In fact they clearly demonstrate that although the starting temperatures of the exothermic phenomenon are similar for the salts of 4-azidobutylamine (FIGS. 1-6) and 4-azidobutylamine (FIG. 7), the energies developed after the exothermic event are considerably lower for 4-azidobutylamine salts. Said salts can therefore easily be transported and used to prepare active pharmaceutical ingredients such as macrolides, preferably solithromycin, and to prepare polymers or used as spacers useful in organic synthesis.

A further subject of the present invention is therefore a salt of a compound of formula (I) with an organic acid for use in the preparation of chemical compounds, and in particular of an active pharmaceutical ingredient, preferably solithromycin.

A further subject of the present invention is a salt of a compound of formula (I) with an organic acid for the preparation of a polymer or a spacer useful in organic synthesis.

The following examples illustrate the invention.

### EXAMPLES

### 1. Preparation of 4-azidobutylamine cholate

512.0 mg of 4-azidobutylamine is dissolved in 8 ml of methyl-tent-butyl-ether. 1.9 g of cholic acid is added to the solution. The suspension is left under stirring for 16 hours at 20-25° C., and the solid is then recovered by filtration through a Büchner funnel. After stove drying at 25° C. for 3-4 hours, 2.16 g of product is obtained. Yield: 92%; purity measured as HPLC Area % (A %): >99.99%. The 4-azidobutylamine cholate salt thus obtained presents a DSC pattern as shown in FIG. 1.

By proceeding similarly, starting with the respective organic acids, the following salts can be obtained: 4-azidobutylamine p-toluenesulphonate and 4-azidobutylamine deoxycholate.

### 2. Preparation of 4-azidobutylamine dibenzoyl-L-tartrate

96.8 mg of 4-azidobutylamine is dissolved in 2 ml of methyl-tent-butyl-ether. 307.0 mg of dibenzoyl-L-tartaric acid is added to the solution. The suspension is left under stirring for 16 hours at 20-25° C., and the solid is then recovered by filtration through a Büchner funnel. After stove drying at 25° C. for 3-4 hours, 300 mg of product is obtained. Yield: 75%; purity measured as HPLC Area % (A %): >99.99%.

The 4-azidobutylamine L-dibenzoyl tartrate salt thus obtained presents a DSC pattern as shown in FIG. 3.

### 3. Preparation of 4-azidobutylamine camphorsulphonate

988.8 mg of 4-azidobutylamine is dissolved in 13 ml of methyl-tert-butyl-ether. 2.0 g of (±)-10-camphorsulphonic acid is added to the solution. The suspension is left under stirring for 16 hours at 20-25° C., and the solid is then recovered by filtration through a Büchner funnel. After stove drying at 25° C. for 3-4 hours, 2.74 g of product is obtained. Yield: 91%; purity measured as HPLC Area % (A %): >99.99%.

The 4-azidobutylamine camphorsulphonate salt thus obtained presents a DSC pattern as shown in FIG. 4.

### 4. Preparation of 4-azidobutylamine 4-phenylbutyl-2-carboxyethyl-phosphinate

96.8 mg of 4-azidobutylamine is dissolved in 2 ml of methyl-tert-butyl-ether. 220.0 mg of 4-phenylbutyl-2-carboxyethyl-phosphinic acid is added to the solution. The suspension is left under stirring for 16 hours at 20-25° C., and the solid is then recovered by filtration through a Büchner funnel. After stove drying at 25° C. for 3-4 hours, 160 mg of product is obtained. Yield: 51%; purity measured as HPLC Area % (A %): >99.99%. The 4-azidobutylamine 4-phenylbutyl-2-carboxyethyl-phosphinate salt thus obtained presents a DSC pattern as shown in FIG. 6.

1-10. (canceled)

11. A salt of a compound of formula (I)
NH₂—(CH₂)n-N₃ (I)
with an organic acid, wherein n is an integer from 1 to 15, in crystalline, amorphous or solvated form.

12. The salt of a compound of formula (I) according to claim 11 wherein n is an integer from 2 to 6.

13. The salt according to claim 11, wherein the compound of formula (I) is 4-azidobutylamine.

14. The salt according to claim 11, wherein the organic acid is selected from the group consisting of a substituted or unsubstituted carboxylic acid, sulphonic acid, phosphinic acid and phosphonic acid.

15. The salt according to claim 11, wherein the carboxylic acid is a monocarboxylic acid, selected from the group consisting of a cholanic acid, pantoic acid, pantothenic acid, folic acid, a fatty acid, glycolic acid, hyaluronic acid, acetylsalicylic acid, salicylic acid; a dicarboxylic acid selected from the group consisting of tartaric acid, dibenzoyltartaric acid, fumaric acid, succinic acid, adipic acid, malic acid, maleic acid, oxalic acid; and a tricarboxylic acid.

16. The salt according to claim 11, wherein the organic acid is selected from the group consisting of cholic acid, deoxycholic acid, dibenzoyltartaric acid, camphorsulphonic acid, para-toluenesulphonic acid and 4-phenylbutyl-2-carboxyethyl-phosphinic acid.

17. The salt according to claim 11, wherein said salt is selected from the group consisting of 4-azidobutylamine cholate, 4-azidobutylamine p-toluenesulphonate, 4-azidobutylamine camphorsulphonate, 4-azidobutylamine deoxycholate, 4-azidobutylamine L-dibenzoyl tartrate and 4-azidobutylamine 4-phenylbutyl-2-carboxyethyl-phosphinate.

18. The salt according to claim 11, wherein said salt is selected from the group consisting of 4-azidobutylamine cholate, 4-azidobutylamine L-dibenzoyl tartrate, 4-azidobutylamine camphorsulphonate and 4-azidobutylamine 4-phenylbutyl-2-carboxyethyl-phosphinate.

19. A process for the preparation of a salt of a compound of formula (I),
NH₂—(CH₂)n-N₃ (I)
wherein n is an integer from 1 to 15, in crystalline, amorphous or solvated form;
comprising:
a) forming a solution of a compound of formula (I) in a solvent;
b) adding an organic acid to the resulting solution to obtain a precipitate; and
c) recovering the salt of a compound of formula (I).

20. A polymer or spacer useful in organic synthesis, wherein said polymer or spacer is made from a salt of a compound of formula (I)
NH₂—(CH₂)n-N₃ (I)
with an organic acid, wherein n is an integer from 1 to 15, in crystalline, amorphous or solvated form.

21. A pharmaceutically active ingredient, wherein said pharmaceutically active ingredient is made from a salt of a compound of formula (I)
NH₂—(CH₂)n-N₃ (I)
with an organic acid, wherein n is an integer from 1 to 15, in crystalline, amorphous or solvated form.

22. A macrolide made using a salt of a compound of formula (I)
NH₂—(CH₂)n-N₃ (I)
with an organic acid, wherein n is an integer from 1 to 15, in crystalline, amorphous or solvated form.

23. The macrolide according to claim 22, wherein the macrolide is solithromycin.

24. The macrolide according to claim 22, wherein the salt of a compound of formula (I) is selected from the group consisting of 4-azidobutylamine cholate, 4-azidobutylamine L-dibenzoyl tartrate, 4-azidobutylamine camphorsulphonate and 4-azidobutylamine 4-phenylbutyl-2-carboxyethyl-phosphinate.

25. The macrolide according to claim 23, wherein the salt is selected from the group consisting of 4-azidobutylamine cholate, 4-azidobutylamine L-dibenzoyl tartrate, 4-azidobutylamine camphorsulphonate and 4-azidobutylamine 4-phenylbutyl-2-carboxyethyl-phosphinate.

26. The salt according to claim 15, wherein the tricarboxylic acid is citric acid.
